(19) United States
(12) Patent Application Publication
(10) Pub. No.: US 2007/0134744 A1
(43) Pub. Date: Jun. 14, 2007

(54) Epothilone resistant cell lines

(76) Inventors: Peter Wisdom Atadja, Parsippany, NJ (US); Markus Wartmann, Riehen (CH); Yan Yan-Neale, Edison, NJ (US); Dalia Cohen, Livingston, NJ (US)

Correspondence Address: NOVARTIS;CORPORATE INTELLECTUAL PROPERTY, ONE HEALTH PLAZA 104/3, EAST HANOVER, NJ, 07936-1080, US

(21) Appl. No.: 11/650,375

(22) Filed: Jan. 5, 2007

Related U.S. Application Data
(63) Continuation of application No. 10/432,072, filed on May 19, 2003, now abandoned.
(60) Provisional application No. 60/252,706, filed on Nov. 22, 2000.

(51) Int. Cl. G01N 33/574; C12N 5/08; A61K 31/427

(52) U.S. Cl. 435/7.23; 435/366; 514/365

(57) ABSTRACT

Epothilone resistant cells lines are disclosed. The invention also discloses methods for identifying substances which are cytotoxic to epothilone resistant cells or which are chemosensitizers or analogs of epothilone. The invention further discloses methods for identifying epothilone resistant cells and for inhibiting the growth of epothilone resistant cells in vitro and in vivo. The invention also discloses antibodies specific for epothilone resistant cells. Also disclosed is a method to identify microtubule stabilizing agents using the epothilone resistant cell lines disclosed.

This invention relates to newly identified epothilone A and epothilone B resistant cell lines.

### BACKGROUND OF THE INVENTION

Multidrug resistance refers to the process whereby cells acquire resistance to certain cytotoxic agents and also demonstrate acquired cross-resistance to other, sometimes structurally and functionally unrelated drugs. Acquired resistance to chemotherapeutic agents in this way is a major obstacle to the effective treatment of cancer. Although the best characterized mechanism of resistance to anticancer drugs involves the overexpression of proteins of the P-glycoprotein (P-gp) and multidrug related protein (MRP) family, which are plasma membrane ATP-dependent pumps that efflux a large variety of cytotoxic drugs out of the cell, not all multidrug resistant cells overexpress these proteins, indicating the existence of non-P-gp/MRP mediated mechanisms of multidrug resistance.

The epothilones, especially epothilones A and B, represent a new class of microtubule stabilizing cytotoxic agents (see Gerth, K. et al., J. Antibiot. 49, 560-3 (1996); or Hoefle et al., DE 41 38 042), e.g. with the formulae: 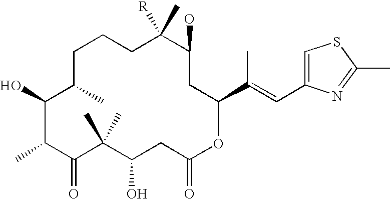
wherein R is hydrogen (epothilone A) or methyl (epothilone B).

The epothilones are macrolide polyketides that, like paclitaxel (TAXOL®), have the ability to arrest cells in mitosis, bind directly to tubulin and preferentially microtubules, cause formation of bundles of intracellular microtubules in non-mitotic cells and induce the formation of hyperstable tubulin polymers. In contrast to TAXOL®, however, epothilones A and B do not appear to be substrates for P-gp, in that they retain activity against P-gp expressing multidrug resistant cancer cell lines. Additionally, epothilone-mediated microtubule stabilization does not trigger endotoxin signaling, an effect that is thought to mediate some non-hematological effects of TAXOL® in cancer therapy (see Muhlardt, P. F. and Sasse, F. (1997) Cancer Research 57:3344-3346).

In addition, epothilones show better water solubility, than TAXOL® and are thus more appropriate for formulation. Since epothilones are not subject to P-gp or MRP mediated multi-drug resistance, they can inhibit the proliferation of cells that otherwise show resistance to treatment with other chemotherapeutics due to the activity of the P-glycoprotein efflux pump; incubation of cells with epothilones induces stabilization of microtubules followed by apoptosis (see Bollag, D. M., et al., “Epothilones, a new class of microtubule-stabilizing agents with a TAXOL®-like mechanism of action”, Cancer Research 55, 2325-33 (1995); and Bollag D. M., Exp. Opin. Invest. Drugs 6, 867-73 (1997)). Furthermore, despite apparently sharing the same, or a sterically proximal binding site on the microtubule, the epothilones have been shown to be active against a TAXOL®-resistant ovarian carcinoma cell line that carry a mutation in a β-tubulin isoform (see Kowalski, R. J., et al., J. Biol. Chem. 272(4), 2534-2541 (1997)).

While two epothilone-resistant human ovarian carcinoma cell lines have been previously described in the literature (Giannakakou et al., PNAS 97(6): 2904-2909 (2000)), data disclosed herein indicate that the cell lines of the present invention are phenotypically distinct from the epothilone-resistant cells previously described. For example, while the cell lines disclosed in Giannakakou et al. display resistance to both epothilone A and B, the EA150 cells disclosed herein appear to exhibit very little resistance to TAXOL® and epothilone B. Similarly, while the EpoA8 cell lines in Giannakakou et al. show epothilone resistance, but limited paclitaxel resistance, both the C5/0 and D4/40 cell lines disclosed herein display full cross-resistance to paclitaxel. Furthermore, the D4/40 cell lines disclosed herein display not only drug resistance but also drug dependency.

In addition, the epothilone-resistant cell lines disclosed in Giannakakou et al. were shown to contain acquired β-tubulin mutations which affect the ability of epothilones to induce tubulin polymerization as well as inhibit cell growth. Both the C5/0 and D4/40 cell lines disclosed herein carry a single point mutation (Threonine at position 274 replaced by Proline) in the HM40 tubulin isoform. While the site of mutation is identical to that reported for the EpoA8 cell line, the nature of the mutation (Threonine at position 274 replaced by Isoleucine) is not. Thus, while a similar mutation may be, in part, involved in the mechanism of resistance seen in the cell lines disclosed herein, phenotypic differences between the cell lines of the instant invention and those described by Giannakakou et al. indicate that the mechanism of action of epothilone resistance and associated genotypic alterations, which remain to be fully characterized, may not be the same in all resistant cell lines. As such, it is believed that the cells lines of the present invention are phenotypically and genotypically distinct from the epothilone resistant cells previously reported.

### SUMMARY OF THE INVENTION

One aspect of the invention provides an epothilone resistant subline of the MDA-MB-435 breast adenocarcinoma cell line. In one embodiment the epothilone resistant cell line is resistant to 10 nM epothilone A and is the EA10 cell line. In another embodiment the epothilone resistant cell line is resistant to 20 nM epothilone A and is the EA20 cell line. In yet another embodiment the epothilone resistant cell line is resistant to 40 nM epothilone A and is the EA40 cell line. In still another embodiment the epothilone resistant cell line is resistant to 60 nM epothilone A and is the EA60 cell line. In another embodiment the epothilone resistant cell line is resistant to 150 nM epothilone A and is the EA150 cell line.

Another aspect of the invention provides epothilone resistant sublines of the KB-31 carcinoma cell line. In one embodiment the epothilone resistant cell line is the carcinoma cell line 297/C5/0. In another embodiment the epothilone resistant cell line is the carcinoma cell line 298/D4/40. In a further embodiment the epothilone resistant cell line is the carcinoma cell line 315/sc5.9.

Another aspect of the invention is a method to identify agents which display improved cytotoxicity to epothilone resistant cells in comparison to epothilone, said method comprising the steps of: incubating said epothilone resistant cells and sensitive cells with an agent to be tested; determining the cytotoxicity of said agent for epothilone resistant cells and sensitive cells; and, identifying agents which display a reduced resistance factor (IC₅₀ value for resistant cells divided by IC₅₀ value for parental cells) compared to epothilone.

A further aspect of the invention provides a method to identify agents which display selective cytotoxicity to epothilone resistant cells in comparison to epothilone, said method comprising the steps of: incubating said epothilone resistant cells and sensitive cells with an agent to be tested; determining the cytotoxicity of said agent for epothilone resistant cells and sensitive cells; and, identifying agents which display a resistance factor (IC₅₀ value for resistant cells divided by IC₅₀ value for parental cells)<1.

In yet another aspect, the invention provides the agents identified according to the methods disclosed herein which are selectively cytotoxic to epothilone resistant cells.

Another aspect of the invention is a method to selectively inhibit the growth of epothilone resistant cells in vitro or in vivo, said method comprising contacting said resistant cells with one or more of said cytotoxic agents identified according to the methods disclosed herein.

Another aspect of the invention is a method to identify agents which are chemosensitizers of epothilones, said method comprising the steps of incubating epothilone resistant cells with an epothilone to which the cells are resistant, in the presence and absence of an agent to be tested and determining the cytotoxicity of the epothilone for the cells, wherein increased cytotoxicity in cultures incubated in the presence of the agent compared to cultures incubated in the absence of the agent indicates that the agent is a chemosensitizer.

In yet another aspect, the invention provides the chemosensitizers of epothilones identified according to the methods disclosed herein.

In a still further aspect, the invention provides a method to inhibit the growth of epothilone resistant cells in vitro or in vivo, said method comprising contacting said epothilone resistant cells with one or more epothilones and one or more of the chemosensitizers identified according to the method disclosed herein.

Another aspect of the invention is a method to identify potential epothilone resistant cells, including tumor cells, said method comprising the steps of preparing total mRNA from test cells, preparing CDNA from the total mRNA of said test cells, performing gene expression analysis of said cDNA samples by conventional methods, for example using chip array techniques; and comparing the expression patterns derived from said test cells to that obtained for epothilone resistant cells, wherein similar patterns of expression suggest that the test cells are potentially epothilone resistant cells. As an extension of this method, conventional methods, for example chip array techniques, could be applied to obtain a differential gene expression pattern by comparing diseased tissue or test cells with non-diseased control tissue or cells, and then compare the pattern of differentially expressed genes with that obtained by comparison of epothilone resistant cells with parental epothilone-sensitive cells. A similar differential gene expression pattern may suggest that the diseased tissue or test cells are potentially resistant to epothilone.

In still another aspect, the invention provides purified antibodies specific for the epothilone resistant cells of the present invention. Additionally, the invention provides pharmaceutical compositions comprising one or more of these purified antibodies in conjunction with a pharmaceutically acceptable carrier or diluent.

In another aspect, the invention provides a method of identifying epothilone resistant cell lines, said method comprising the steps of labelling one or more antibodies disclosed herein, exposing test cells to said one or more labelled antibodies; and measuring binding of said one or more labelled antibodies thereto.

In a further aspect, the invention provides a method of isolating epothilone resistant cell lines, said method comprising labelling one or more antibodies disclosed herein with a fluorescent dye;
incubating cell mixtures with said one or more fluorescently labelled antibodies; and,
isolating epothilone resistant cells fluorescently labelled with one or more antibodies by use of a fluorescence activated cell sorter. An alternative method of separating antibody-reactive epothilone-resistant cells from normal, non-reactive cells would be to covalently link one or more claimed antibodies with magnetic beads and then to isolate epothilone resistant cells by immobilizing them using a magnetic source while washing away unbound, non-reactive cells.

In another aspect, the invention provides a method for killing epothilone resistant cells, said method comprising attaching a substance having cytotoxic or therapeutic activity to one or more antibodies specific for epothilone-resistant cells; administering said one or more antibodies to cell culture (or a patient); and, targeting the cytotoxic drug to epothilone resistant cells, selectively killing these cells.

In yet another aspect, the invention provides a method for diagnosing an epothilone resistant cancer in vivo, comprising labelling one or more of the antibodies disclosed herein, administering said one or more labelled antibodies to a patient or a patient tissue specimen; and identifying tissue to which said one or more labelled antibodies bind.

In a further aspect, the invention provides a method of treating cancer, said method comprising administering to a subject in need thereof a therapeutically effective amount of one or more of the antibodies specific for epothilone-resistant cells.

In another aspect, the invention provides a method to identify microtubule-binding agents with a mechanism of action similar to that of epothilone A or B, said method comprising the steps of contacting an epothilone resistant cell line of the present invention and the corresponding parental cell line with increasing concentrations of a test substance; and, determining resistance of the epothilone resistant cell line to the test substance compared to resistance of the parental cell line to the test substance wherein greater resistance to the test substance of the epothilone resistant cell line compared to the parental cell line suggests that the test substance is a microtubule-binding agent that Interacts with tubulin in a fashion similar to epothilone A or B. The thus identified agents could include, but do not have to be restricted to, epothilone analogs.

In still another aspect, the invention provides microtubule-binding agents, including epothilone analogs, identified according to the method disclosed above.

In another aspect, the invention provides a method to identify compounds which are potential microtubule-stablizing agents, said method comprising the steps of incubating epothilone-dependent cells with a test compound wherein said resistant cells are epothilone deprived; and assaying for growth of said dependent cells, wherein growth of said dependent cells would indicate that said compound is a potential microtubule-stabilizing agent. The microtubule stabilizing agents identified according to the method disclosed herein are also herein included as an aspect of the invention.

Yet another aspect of the invention provides a method to identify potential epothilone resistant cells, including tumor cells, said method comprising the steps of using conventional molecular biological techniques to identify cells containing the point mutation characteristic of the epothilone resistant cells disclosed herein.

In a further aspect the invention provides a method to prevent the translation of the mutated form of beta tubulin and thus prevent epothilone resistance in a tumor cell using nucleic acids that are antisense to a region of nucleic acid which contains the point mutation characteristic of the epothilone resistant cells disclosed herein.

### DETAILED DESCRIPTION OF THE INVENTION

The following abbreviations used throughout the disclosure are listed below:
P-gp=P-glycoprotein
MDR=multidrug resistance
MRP=MDR-associated protein
MEM=minimal essential medium
MTS=3-(4,5-Dimethylthiazol-2-yl)-5-(3-carboxymethyoxyphenyl)-2-(4-sulfophenyl)-2H-tetrazolium, inner salt
PMS=phenazine methosulfate

This invention generally relates to epothilone A and epothilone B resistant cells lines. Different aspects of the invention relate to antibodies and methods involving inhibiting and/or identifying epothilone resistance. The various aspects of the invention will be described in the following subsections.

1. Epothilone Resistant Cell Lines

Methods for the selection of drug resistant cell lines are known in the art and generally involve culturing and subculturing cells in the presence of increasing concentrations of a drug. Surviving colonies of cells are further expanded in the presence of higher concentrations of drug which eventually results in individual resistant cell lines and sublines of cells (see, for example Akiyama et al., Somat. Cell. Mol. Genetics 11:117-126 (1985)). As used herein “resistance” of a cell to an agent refers to the ability of the cell to tolerate higher concentrations of a drug than a sensitive cell. Thus, epothilone resistance of a cell is determined relative to an appropriate epothilone sensitive cell. For example, the epothilone A resistance of a cell that has been continually exposed to the drug can be determined relative to the parental sensitive cell from which the drug resistant cell was derived. Resistance of a cell to an agent (e.g. epothilone A) is typically quantitated as the increase in IC₅₀ (concentration of the agent needed to inhibit cell growth by 50%) relative to a control sensitive cell. As used herein, the term “epothilone resistance” refers to resistance to epothilone A, epothilone B, or both.

By the term “epothilone”, any epothilone or epothilone derivative is meant. Preferably, the term “epothilone” means epothilone A, epothilone B, any epothilone derivative disclosed in PCT publication WO 98/25929, or any mixture thereof; more preferably, it means epothilone A and epothilone B whose chemical structures are provided above.

As used herein, “test cells” refers to cells that may or may not be epothilone resistant.

Several epothilone resistant cell lines disclosed herein were obtained from culturing and subculturing MDA-MB-435 cell lines in the presence of increasing concentrations of epothilone A. The MDA-MB435 cell line is a breast adenocarcinoma cell line and may be obtained from the ATCC (Manassas, Va., USA). This cell line may be grown in monolayer using Minimal Essential Medium (MEM) supplemented with 10% fetal calf serum, 1% L-glutamine, penicillin (50 Units/ml) and streptomycin (50 μg/ml) and maintained at 37° C. in 5.0% CO₂. Media and supplements are all available from Gibco Life Technologies, Rockville, Md. Cells display a doubling time of about 20 hours and a relative plating efficiency of approximately 60%.

The epothilone resistant EA10 subline disclosed herein is derived from incubating the parental MDA-MB435 breast adenocarcinoma cell line as above in 10 nM of epothilone A. As used herein, the term “subline” (or “subclone”) refers to a cell line that is derived from a parental cell line by virtue of its resistance to one or more epothilones. The epothilone resistant cell lines designated herein as EA20, EA40, EA60 and EA 150 are selected from the EA10 cell line by continued incubation as above in 20 nM, 40 nM, 60 nM and 150 nM epothilone A, respectively. It is intended that the invention encompasses other epothilone A resistant cell lines that may be selected by culturing cells of the MDA-MB-435 breast adenocarcinoma cell line in the presence of other concentrations of epothilone A. The increase in fold resistance of the cell to epothilone can be assessed relative to the parental cell line from which the resistant cell line was derived (e.g., IC₅₀ of epothilone A for the resistant cell line versus IC₅₀ of epothilone A for the parental cell line). The EA20, EA40, EA60 and EA150 epothilone resistant cell lines of the present invention have been deposited on Nov. 21, 2000 with the American Type Culture Collection (ATCC), Manassas, Va. 20110-2209 USA, in compliance with the provisions of the Budapest Treaty on the International Recognition of the Deposit of Microorganisms for the Purposes of Patent Procedure, and have been assigned Deposit Nos. PTA-2702, PTA-2708, PTA-2703 and PTA-2706, respectively.

Additional epothilone resistant cell lines of the present invention were selected by culturing and subculturing the KB-31 human epidermoid carcinoma cell line. KB-31 cells are a subclone of the KB epidermoid carcinoma cell line which, like the MDA-MB-435 cell line discussed above, may be obtained from the ATCC (ATCC number CCL-17). Human epidermoid carcinoma cell lines KB-31 and KB-8511, a P-gp overexpressing MDR cell line derived from KB-31 cells, were originally obtained from Dr. R. M. Baker, Roswell Park Memorial Institute (Buffalo, N.Y.) (for description see Akiyama et al., Somat. Cell. Mol. Genetics 11:117-126 (1985) and Fojo, A. et al., Cancer Res. 45:3002-3007 (1985)) and are cultured as previously described (Meyer, T. et al., Int. J. Cancer 43:851-856 (1989)). The KB-8511 cell line was deposited under the Budapest Treaty on Feb. 20, 1998 at the Deutsche Sammlung von Mikroorganismen und Zellkulturen GmbH (DSMZ, Mascheroder Weg 1b, D-38124 Braunschweig, Germany) under the accession number DSM ACC2342. Applicants have also deposited the parental KB-31 cell line disclosed herein with the ATCC on Nov. 21, 2000 under the Budapest Treaty. This cell line has been given the Patent Deposit Designation number PTA-2701.

KB-31 cells can be grown in monolayer using Minimal Essential Medium alpha (MEMalpha) with 5% fetal bovine serum, L-glutamine (1%, v/v). Penicillin (50 Units/ml) and streptomycin (50 μg/ml) can also be added (Animed, Basel, Switzerland). The cells display a doubling time of about 22 hours. KB-8511, derived from the KB-31 cell line by colchicine treatment, displays about a 40-fold relative resistance against colchicine compared to the KB-31 cells; they can be grown under the same conditions as KB-31 cells. In order to maintain a high level of P-gp expression during prolonged tissue culture, demecolcine (50 ng/ml) can be added to the medium of stock cultures (Sigma, St. Louis, Mo.). While the KB cell line was originally thought to be derived from an epidermal carcinoma of the mouth, isoenzyme analysis, HeLa marker chromosomes and DNA fingerprinting indicate that the parental KB cell line was possibly established due to HeLa cell contamination. Additional details and related references concerning the characteristics of the KB cell line may be found at the ATCC website, http://www.atcc.org.

As explained in detail in the Examples, below, epothilone A and B resistant cell lines were derived by culturing KB-31 cells in increasing concentrations of the drug. The resulting cell lines designated 298/D4/40 (KB-31/298), 297/C5/0 (KB-31/297) and 315/sc5.9 (KB-31/315) have been deposited with the ATCC on Nov. 21, 2000, In compliance with the provisions of the Budapest Treaty, and have been assigned Deposit Nos. PTA-2705; PTA-2704; PTA-2707, respectively.

It is intended that the invention encompasses other epothilone resistant cell lines that may be selected by culturing cells of the KB cell line lineage in the presence of other concentrations of epothilones.

II. Antibodies

Another aspect of the invention are antibodies generated using the epothilone resistant cell lines disclosed herein. Preferably, the antibodies are monoclonal antibodies which bind to one or more cell lines disclosed herein but (1) do not bind to healthy tissue and/or (2) do not bind to parental cell lines. Such antibodies may include, but are not limited to polyclonal antibodies, monoclonal antibodies (mAbs), humanized (i.e., antibody molecules in which the amino acid sequence in the non-antigen binding regions has been altered so that the antibody more closely resembles a human antibody, and still retains its original binding ability), chimeric antibodies, single chain antibodies, Fab fragments, F(ab′)₂ fragments, fragments produced by a Fab expression library, anti-idiotypic (anti-Id) antibodies, and epitope-binding fragments of any of the above. Such antibodies are useful (1) as diagnostic tools, to identify resistant cell lines and (2) as therapeutic agents when coupled to a substance having cytotoxic or therapeutic activity, to treat patients having epothilone resistant cancers. As used herein, the term “substance having a cytotoxic or therapeutic activity” refers to molecules whose action can destroy a cell, such as a radioactive isotope, a toxin (e.g. diptheria toxin or ricin) or a chemotherapeutic drug, as well as cells whose action can destroy another cell, such as cytotoxic cells. The term “cytotoxic cells” includes such cells as macrophages, neutrophils, eosinophils, NK cells, LAK cells and large granular lymphcytes. It is contemplated that the antibody can be coupled to a cytotoxic cell through Fc receptors on the cytotoxic cells. The molecule binding to the epothilone resistant cell can be directly coupled to a substance having a cytotoxic or therapeutic activity (e.g. a ricin-linked monoclonal antibody) or may be indirectly linked to the substance. For example, a bispecific antibody which is capable of crosslinking to a tumor cell and a cytotoxic cell can be used, thereby facilitating lysis of the tumor cell. A bispecific antibody can crosslink a tumor cell and the cytotoxic cell by binding to the Fc receptors of cytotoxic cells.

The invention includes methods for the use of the antibodies of the present invention to identify, isolate and/or kill epothilone resistant cells and (b) in the manufacture of a medicament for treatment of an epothilone resistant cancer; pharmaceutical compositions comprising such an antibody in combination or association with a pharmaceutically acceptable carrier or diluent.

Polyclonal antibodies are heterogeneous populations of antibody molecules derived from the sera of animals immunized with an antigen, such as target gene product, or an antigenic functional derivative thereof. For the production of polyclonal antibodies, various hosts including, but not limited to, goats, rabbits, rats, mice, humans and others may be immunized by injection with live cells, whole cell lysates, partially purified lysates from epothilone resistant cells or proteins preferentially expressed in epothilone resistant cells. Rats and mice are preferred hosts for downstream applications involving monoclonal antibody production. Depending on the host species, various adjuvants may be used to increase immunological response. Such adjuvants include, but are not limited to, Freund's (incomplete or complete), mineral gels such as aluminum hydroxide and surface active substances such as lysolecithin, pluronic polyols, polyanions, peptides, oil emulsions, KLH (keyhole limpet hemocyanin) and dinitrophenol. Among adjuvants used in humans, BCG (bacilli Calmette-Guerin) and Cornybacterium Parvum are especially preferable. (For review of methods for antibody production and analysis, see, e.g., Harlow, E. and Lane, D. (1988) Antibodies: A Laboratory Manual, Cold Spring Harbor Laboratory, Cold Spring Harbor, N.Y.).

Monoclonal antibodies, which are homogeneous populations of antibodies to a particular antigen, may be obtained by any technique which provides for the production of antibody molecules by continuous cell lines in culture. These include, but are not limited to the hybridoma technique of Kohler and Milstein, (1975, Nature 256:495-497; and U.S. Pat. No. 4,376,110), the human B-cell hybridoma technique (Kosbor et al., 1983, Immunology Today 4:72; Cole et al., 1983, Proc. Natl. Acad. Sci. USA 80:2026-2030), and the EBV-hybridoma technique (Cole et al., 1985, Monoclonal Antibodies And Cancer Therapy, Alan R. Liss, Inc., pp. 77-96). Such antibodies may be of any immunoglobulin class including IgG, IgM, IgE, IgA, IgD and any subclass thereof. The hybridoma producing the mAb of this invention may be cultivated in vitro or in vivo. Production of high titers of mAbs in vivo makes this the presently preferred method of production.

In addition, techniques developed for the production of “chimeric antibodies” (Morrison et al., 1984, Proc. Natl. Acad. Sci., 81:6851-6855; Neuberger et al., 1984, Nature, 312:604-608; Takeda et al., 1985, Nature, 314:452-454) by splicing the genes from a mouse antibody molecule of appropriate antigen specificity together with genes from a human antibody molecule of appropriate biological activity can be used. A chimeric antibody is a molecule in which different portions are derived from different animal species, such as those having a variable or hypervariable region derived from a murine mAb and a human immunoglobulin constant region.

Alternatively, techniques described for the production of single chain antibodies (U.S. Pat. No. 4,946,778; Bird, 1988, Science 242:423-426; Huston et al., 1988, Proc. Natl. Acad. Sci. USA 85:5879-5883; and Ward et al., 1989, Nature 334:544-546) can be adapted to produce differentially expressed gene-single chain antibodies. Single chain antibodies are formed by linking the heavy and light chain fragments of the Fv region via an amino acid bridge, to produce single chain antibodies specific for epothilone resistant cells. Antibodies with related specificity, but of distinct idiotypic composition, may be generated by chain shuffling from random combinatorial immunoglobulin libraries. (See, e.g., Burton, D. R. (1991) Proc. Natl. Acad. Sci. 88:10134-10137).

Most preferably, techniques useful for the production of “humanized antibodies” can be adapted to produce antibodies to the polypeptides, fragments, derivatives, and functional equivalents disclosed herein. Such techniques are disclosed in U.S. Pat. Nos. 5,932,448; 5,693,762; 5,693,761; 5,585,089; 5,530,101; 5,910,771; 5,569,825; 5,625,126; 5,633,425; 5,789,650; 5,545,580; 5,661,016; and 5,770,429.

Antibodies may also be produced by inducing in vivo production in the lymphocyte population or by screening immunoglobulin libraries or panels of highly specific binding reagents as disclosed in the literature. (See, e.g., Orlandi, R. et al. (1989) Proc. Natl. Acad. Sci. 86:3833-3837; and Winter, G. et al. (1991) Nature 349:293-299).

Antibody fragments which recognize specific epitopes may be generated by known techniques. For example, such fragments include but are not limited to: the F(ab′)₂ fragments which can be produced by pepsin digestion of the antibody molecule and the Fab fragments which can be generated by reducing the disulfide bridges of the F(ab′)₂ fragments. Alternatively, Fab expression libraries may be constructed (Huse et al., 1989, Science, 246:1275-1281) to allow rapid and easy identification of monoclonal Fab fragments with the desired specificity.

The present invention allows the skilled artisan to prepare bispecific antibodies and tetrameric antibody complexes which bind to a cell line of the invention. Bispecific antibodies can be prepared by forming hybrid hybridomas. The hybrid hybridomas can be prepared using the procedures known in the art such as those disclosed in Staerz and Bevan, PNAS USA (1986) 83:1453 and Immunology Today (1986) 7:241. In general, a hybrid hybridoma is formed by fusing a first cell line which produces a first monoclonal antibody which is capable of binding to a cell line of the present invention and a second cell line which produces a second monoclonal antibody which is capable of binding to a detectable substance, or a substance having toxic or therapeutic activity. The bispecific antibodies can also be constructed using chemical means using procedures previously described in Staerz et al. Nature (1985) 314:628 and Perez et al. Nature (1985) 316:354. Tetrameric antibody complexes may be prepared according to methods familiar to one of skill in the art, for example, according to the teachings found in U.S. Pat. No. 4,868,109.

Various immunoassays may be used for screening to identify antibodies having the desired specificity and minimal cross-reactivity. Numerous protocols for competitive binding or immunoradiometric assays using either polyclonal or monoclonal antibodies with established specificities are well known in the art. Such immunoassays typically involve the measurement of complex formation between epothilone resistant cells or proteins preferentially expressed in epothilone resistant cells and its specific antibody.

Antibody binding affinity can be determined by conventional methods, including Scatchard analysis in conjunction with radioimmunoassay techniques.

Particularly preferred, for ease of detection, is the sandwich assay, of which a number of variations exist, all of which are intended to be encompassed by the present invention. For example, in a typical forward assay, unlabeled antibody is immobilized on a solid substrate and the sample to be tested brought into contact with the bound molecule. After a suitable period of incubation, for a period of time sufficient to allow formation of an antibody-antigen binary complex. At this point, a second antibody, labeled with a reporter molecule capable of inducing a detectable signal, is then added and incubated, allowing time sufficient for the formation of a ternary complex of antibody-antigen-labeled antibody. Any unreacted material is washed away, and the presence of the antigen is determined by observation of a signal, or may be quantitated by comparing with a control sample containing known amounts of antigen. Variations on the forward assay include the simultaneous assay, in which both sample and antibody are added simultaneously to the bound antibody, or a reverse assay in which the labeled antibody and sample to be tested are first combined, incubated and added to the unlabeled surface bound antibody. These techniques are well known to those skilled in the art, and the possibility of minor variations will be readily apparent. As used herein, “sandwich assay” is intended to encompass all variations on the basic two-site technique. For the immunoassays of the present invention, the only limiting factor is that the labeled antibody be an epothilone resistant cell-specific antibody.

The most commonly used reporter molecules in this type of assay are either enzymes, fluorophore- or radionuclide-containing molecules. In the case of an enzyme immunoassay, an enzyme is conjugated to the second antibody, usually by means of glutaraldehyde or periodate. As will be readily recognized, however, a wide variety of different ligation techniques exist, which are well-known to the skilled artisan. Commonly used enzymes include horseradish peroxidase, glucose oxidase, beta-galactosidase and alkaline phosphatase, among others. The substrates to be used with the specific enzymes are generally chosen for the production, upon hydrolysis by the corresponding enzyme, of a detectable color change. For example, p-nitrophenyl phosphate is suitable for use with alkaline phosphatase conjugates; for peroxidase conjugates, 1,2-phenylenediamine or toluidine are commonly used. It is also possible to employ fluorogenic substrates, which yield a fluorescent product rather than the chromogenic substrates noted above. A solution containing the appropriate substrate is then added to the tertiary complex of antibody-epothilone resistant cell specific antigen-labeled antibody. The substrate reacts with the enzyme linked to the second antibody, giving a qualitative visual signal, which may be further quantitated, usually spectrophotometrically, to give an evaluation of the amount of epothilone resistant cell specific antigen which is present in the serum sample.

In order to probe for the potential presence of the particular single-nucleotide mutation identified in the C5/0 and D4/40 cells, test cell lines, tumors or serum samples of patients can be analysed by mutation detection techniques known to the molecular biologist skilled in the art. These techniques may include SSO (use of sequence-specific oligonucleotides), PASA (PCR amplification of specific alleles), solid-phase minisequencing, multiplex solid-phase fluorescent primer extension, OLA (dual-color oligonucloetide ligation assay), LCR (ligase chain reaction) and UHG (universal heteroduples generator) analyis (for a summary of these methods see Landgren (1996) “Laboratory Protocols for Mutation Detection”, Oxford University Press, ISBN 0 19 857795 8).

Alternately, fluorescent compounds, such as fluorescein and rhodamine, may be chemically coupled to antibodies without altering their binding capacity. When activated by illumination with light of a particular wavelength, the fluorochrome-labeled antibody absorbs the light energy, inducing a state of excitability in the molecule, followed by emission of the light at a characteristic longer wavelength. The emission appears as a characteristic color visually detectable with a light microscope. Immunofluorescence and EIA techniques are both very well established in the art and are particularly preferred for the present method. However, other reporter molecules, such as radioisotopes, chemiluminescent or bioluminescent molecules may also be employed. It will be readily apparent to the skilled artisan how to vary the procedure to suit the required use.

An additional embodiment of the invention relates to the administration of a pharmaceutical composition, in conjunction with a pharmaceutically acceptable carrier, for any of the therapeutic effects discussed herein. Such pharmaceutical compositions may include, but are not limited to, antibodies to epothilone resistant cell specific antigens. The compositions may be administered alone or in combination with at least one other agent, such as stabilizing compound, which may be administered in any sterile, biocompatible pharmaceutical carrier, including, but not limited to, saline, buffered saline, dextrose, and water. The compositions may be administered to a patient alone, or in combination with other agents, including other chemotherapeutic agents, drugs or hormones.

As used herein, the term “other chemotherapeutic agent” refers especially to any chemotherapeutic agent that is or can be used in the treatment of tumor diseases, such as chemotherapeutics derived from the following classes:
(A) Alkylating agents, preferably cross-linking chemotherapeutics, preferably bis-alkylating agents;
(B) antitumor antibiotics, preferably doxorubicin (ADRIAMYCIN®, RUBEX®);
(C) antimetabolites;
(D) plant alkaloids;
(E) hormonal agents and antagonists;
(F) biological response modifiers, preferably lymphokines or interferons;
(G) inhibitors of protein tyrosine kinases and/or serine/threonine kinases;
(H) antisense oligonucleotides or oligonucleotide derivatives;
(I) microtubule stablizers/destabilizers; or
(J) miscellaneous agents or agents with other or unknown mechanism of action.

The pharmaceutical compositions encompassed by the invention may be administered by any number of routes including, but not limited to oral, intravenous, intramuscular, intra-articular, intra-arterial, intramedullary, intrathecal, intraventricular, transdermal, subcutaneous, intraperitoneal, intranasal, enteral, topical, sublingual, or rectal means.

In addition to the active ingredients, these pharmaceutical compositions may contain pharmaceutically acceptable carriers or diluents comprising excipients and auxiliaries which facilitate processing of the active compounds into preparations which can be used pharmaceutically. The term “pharmaceutically acceptable carrier or diluent” includes, but is not limited to, fillers, such as sugars, for example lactose, saccharose, mannitol or sorbitol, cellulose preparations and/or calcium phosphates, for example tricalcium phosphate or calcium hydrogen phosphate, and binders, such as starch pastes using for example corn, wheat, rice or potato starch, gelatin, tragacanth, methylcellulose, hydroxypropylmethyl-cellulose, sodium carboxymethylcellulose and/or polyvinylpyrrolidone, and/or, if desired, disintegrators, such as the above-mentioned starches, also carboxymethyl starch, crosslinked polyvinylpyrrolidone, agar, alginic acid or a salt thereof, such as sodium alginate. Excipients are especially flow conditioners and lubricants, for example silicic acid, talc, stearic acid or salts thereof, such as magnesium or calcium stearate, and/or polyethylene glycol. Dragée cores are provided with suitable, optionally enteric, coatings, there being used, inter alia, concentrated sugar solutions which may comprise gum arabic, talc, polyvinylpyrrolidone, polyethylene glycol and/or titanium dioxide, or coating solutions in suitable organic solvents, or, for the preparation of enteric coatings, solutions of suitable cellulose preparations, such as ethylcellulose phthalate or hydroxypropylmethylcellulose phthalate. Capsules are dry-filled capsules made of gelatin and soft sealed capsules made of gelatin and a plastiziser, such as glycerol or sorbitol. The dry-filled capsules may comprise the active ingredient in the form of granules, for example with fillers, such as lactose, binders, such as starches, and/or glidants, such as talc or magnesium stearate, and if desired with stabilizers. In soft capsules the active ingredient is preferably dissolved or suspended in suitable oily excipients, such as fatty oils, paraffin oil or liquid polyethylene glycols, and also stabilizers and/or antibacterial agents may be added. Dyes or pigments may be added to the tablets or dragée coatings or the capsule casings, for example for identification purposes or to indicate different doses of active ingredient.

As used herein, “therapeutically effective amount” refers to that amount of active ingredient, for example an amount of antibodies raised against the epothilone resistant cell lines disclosed herein which when coupled to a substance having toxic or therapeutic activity, can ameliorate a symptom or condition e.g. cause the death of a tumor cell. Therapeutic efficacy and toxicity may be determined by standard pharmaceutical procedures in cell cultures or experimental animals, e.g., ED50 (the dose therapeutically effective in 50% of the population) and LD50 (the dose lethal to 50% of the population). The dose ratio between toxic and therapeutic effects is the therapeutic index, and it can be expressed as the ratio, LD50/ED50. Pharmaceutical compositions which exhibit large therapeutic indices are preferred. The data obtained from cell culture assays and animal studies is used in formulating a range of dosage for human use. The dosage contained in such compositions is preferably within a range of circulating concentrations that include the ED50 with little or no toxicity. The dosage varies within this range depending upon the dosage form employed, sensitivity of the patient, and the route of administration.

For any compound, the therapeutically effective amount can be estimated initially either in cell culture assays, e.g., of neoplastic cells, or in animal models, usually mice, rabbits, dogs, or pigs. The animal model may also be used to determine the appropriate concentration range and route of administration. Such information can then be used to determine useful doses and routes for administration in humans.

The exact dosage will be determined by the practitioner, in light of factors related to the subject that requires treatment. Dosage and administration are adjusted to provide sufficient levels of the active moiety or to maintain the desired effect. Factors which may be taken into account include the severity of the disease state, general health of the subject, age, weight, and gender of the subject, diet, time and frequency of administration, drug combination(s), reaction sensitivities, and tolerance/response to therapy. Long-acting pharmaceutical compositions may be administered every 3 to 4 days, every week, or once every two weeks, or once every three weeks, depending on half-life and clearance rate of the particular formulation.

Guidance as to particular dosages and methods of delivery is provided in the literature and generally available to practitioners in the art. Those skilled in the art will employ different formulations for nucleotides than for proteins or their inhibitors. Similarly, delivery of polynucleotides or polypeptides will be specific to particular cells, conditions, locations, etc. Pharmaceutical formulations suitable for oral administration of proteins are described, e.g., in U.S. Pat. Nos. 5,008,114; 5,505,962; 5,641,515; 5,681,811; 5,700,486; 5,766,633; 5,792,451; 5,853,748; 5,972,387; 5,976,569; and 6,051,561. In the case of combinations with other chemotherapeutic agent(s), the other chemotherapeutic agents(s) is/are used in standard formulations that are marketed and known to the person of skill in the art.

Further details on techniques for formulation and administration may be found in the latest edition of Remington's Pharmaceutical Sciences (Maack Publishing Co., Easton, Pa.).

III. Methods of the Invention

One aspect of the present invention pertains to methods for indentifying, isolating and/or killing epothilone resistant cells, including tumor cells, using the antibodies disclosed above. For example, a detection method may involve contacting a test cell with an antibody directed to a protein expressed inside or, preferentially, on an epothilone resistant cell disclosed herein. In this method, the antibody is labeled with a detectable substance. Such detectable substances include fluorescent marker, enzyme or radioactive marker which can be used to identify an epothilone resistant tumor cell in vitro or in a tumor sample ex vivo or in vivo. An epothilone resistant tumor cell can be identified by incubating an antibody of the invention with a tumor cell to be tested for epothilone resistance. If the antigen recognized by the epothilone-resistance specific antibody should be known to be expressed inside the cell, the cells would have to be permeabilzed or fixed prior to the administration of the antibody. Binding of the antibody to the tumor cell is indicative of the presence on the tumor cell of a protein expressed inside or on the epothilone resistant cell. The level of antibody binding to the tumor cell can be compared to the level of antibody binding to a normal control cell, and increased binding of the antibody to the tumor cell as compared to the normal cell can be used an indicator of epothilone resistance. Binding of the antibody to a cell (e.g. a tumor cell to be tested or a normal control cell) is determined by detecting the detectable substance with which the antibody is labeled. The detectable substance may be directly coupled to the antibody or alternatively, the detectable substance may be coupled to another molecule which can bind the antibody. For example, a second antibody directed to the first antibody, wherein the second is coupled to a detectable substance.

Epothilone resistant tumor cells can be detected in a tumor sample in vitro or in vivo. For example, tumor tissue removed from a patient can be used as the tumor sample. A sample can be used immediately or frozen and stored at termperature below −20° C. for later use. A tumor section on a microscope slide can be reacted with antibodies using standard immunohistochemistry techniques or with nucleic acids by standard in situ hybridization techniques. Additionally, if a single cell suspension of tumor cells is acheivable, tumor cells can be reacted with antibody and analyzed by flow cytometry. Alternatively, an epothilone resistant tumor cell can be detected in vivo in a subject bearing a tumor. Labelled antibodies can be introduced into the subject and antibodies bound to the tumor can be detected. For example, the antibody can be labeled with a radioactive marker whose presence and location in a subject can be detected by standard imaging techniques.

Reagents useful for identifying an epothilone resistant tumor, for example, antibodies of the present invention, can be incorporated into a diagnostic kit. The kit can contain standards to which a sample is compared. The various reagents can be included in the kit in suitable containers and the kit can include a holder for the containers as well as an instruction manual for the use of the kit.

A tumor section or plasma from a patient could be analysed using molecular probes designed based on the mutation detection techniques described above or according to other conventional methods. In addition, it is contemplated that an antisense nucleic acid directed to a region of nucleic acid containing the point mutation characteristic to the epothilone A resistant cells disclosed herein could be created using molecular biological techniques familiar to one of skill in the art. For a discussion of the regulation of gene expression using antisense genes, see Weintraub, H. et al. Antisense RNA as a molecular tool for genetic analysis, Reviews-Trends in Genetics, Vol. 1(1) 1986. The antisense oligonucleotides could be used in a method for inhibiting expression of the nucleic acid (e.g. mRNAs) encoding the protein which confers on the cells epothilone resistance. Decreasing expression of this protein can be used a means to inhibit or reverse the epothilone resistance of a cell into which the antisense nucleic acid has been introduced. The antisense oligonucleotides could be added to epothilone resistant cells in culture to create cells in which resistance has been inhibited. Such cells in vitro could be used to test potential therapeutic agents. It is also contemplated that the antisense oligonucleotides could be used in gene therapy to reverse or prevent epothilone resistant cancers in a subject. This could be done using conventional techniques for delivery of nucleic acids in vivo. Many references exist regarding the use and administration of pharmaceutical compounds and formulations which include antisense nucleic acids are familiar to one of skill in the art. (See for example, U.S. Pat. Nos. 6,124,133, 6,096,543, and 6,117,848).

The invention further provides a method to identify epothilone resistant cells, including tumor cells, involving the use of cDNA microarrays or other conventional methodology to perform differential gene expression analysis of cDNA prepared from total mRNA pools of test cells, epothilone resistant samples and corresponding reference samples (non-diseased tissue control tissue and epothilone-sensitive cells). Identification of potential epothilone resistant cells is based upon obtaining a similar pattern of genes differentially expressed in test cells and the epothilone resistant cell lines of the present invention, versus their corresponding controls. Differential gene expression can be performed using cDNA generated from RNA isolated from cell samples from in vitro or in vivo sources using conventional methods. Total and messenger RNA preparation, Northern analysis and differential gene expression analysis may be performed according to any of the methods well known in the art.

By the same token, one could also perform proteome analysis (2-D gel electrophoresis of cellular proteins) to identify resistance-associated protein patterns (“fingerprints”). Specifically, epothilone resistant and sensitive parental cells could be analyzed by proteomic techniques in order to identify a differential protein expression pattern associated with epothilone-resistant cells. Thus, protein extracts from the cells could be resolved by two-dimensional gel electrophoresis (first dimension isoelectric focusing and second dimension SDS polyacrylamide gel electrophoresis) according to conventional methods. The expressed proteins could then be visualized by silver-staining or any other conventional protein-stain method and the patterns compared between the two cell lines. The differential protein expression pattern typifying the resistant cell line could be used as a “finger print” potentially identifying other putative epothilone-resistant cells.

The invention further provides a method for identifying a substance that is a chemosensitizer of epothilones. As used herein, the term “chemosensitizer of epothilones” refers to a substance that can increase the efficacy of a therapeutic agent against a resistant cell and/or decrease the resistance of a cell for a therapeutic agent. For example, verapamil is a chemosensitizer of P-gp-mediated multidrug resistance; in the presence of verapamil, a multidrug resistant cell is more susceptible to the cytotoxic effect of anthracyclines. The cell lines of the present invention are particularly useful in a method to identify substances that are chemosensitizers of epothilones. This could comprise the steps of incubating the epothilone resistant cells with an epothilone to which the cells are resistant in the presence and absence of an agent to be tested; and determining the cytotoxicity of the epothilone for the cells, wherein increased cytotoxicity in cultures incubated with the agent compared to cultures incubated in the absence of the agent indicates that the agent is a chemosensitizer. Cytotoxicity can be measured using methods known to one of skill in the art, for example, a colorimetric assay system such as CELLTITER 96® AQUEOUS assay (PROMEGA, Madison, Wis.).

The invention also provides a method for identifying epothilone analogues. As used herein, the term “epothilone analog” refers to a synthetic or natural compound that possesses epothilone-like characteristics. The term may include, but is not limited to, microtubule stabilizing agents. As contemplated herein, the cell lines of the present invention can be used to identify epothilone analogues that may partially overcome the resistance of cell lines for epothilohes. For example, one could incubate an epothilone resistant cell line and corresponding parental cell line with increasing concentrations of a test substance and then determine the relative resistance factor by dividing the IC₅₀ determined for the resistant cell line by that determined for the parental cell line. A test compound with a decreased resistance factor (compared to the epothilone included in the same assay as a reference) would constitute a potential analogue of interest. For example, one could incubate 297/C5/0 cells as well as parental KB-31 cells with increasing concentrations of a test substance and then determine the relative resistance factor by dividing the IC₅₀ determined for 297/C5/0 by that determined for KB-31 cells. A test compound with a decreased resistance factor (compared to epothilone A included in the same assay as a reference) would constitute a potential hit. In addition, experiments with the 298/D4/40 cell line disclosed herein (Which is not only epothilone resistant but also epothilone dependent) would be useful to identify compounds with a mechanism of action similar to that of epothilone; for example, using conventional methodologies, one could screen various libraries for drugs that support the growth of these cells as a way to identify an agent with a similar mechanisms of action as epothilone A.

The invention also provides a method for identifying substances that are selectively cytotoxic to epothilone resistant cells. As used herein, the term “cytotoxic agent” refers to compounds, including anticancer drugs, that inhibit cell proliferation and induce cell death. The method could comprise incubating the resistant cell lines disclosed herein with the substance to be tested and, using standard methods, determining the cytotoxicity of the substance for the resistant cell line and comparing it to that of the parental cell line. Compounds that preferentially kill epothilone-resistant cell lines, i.e. compounds where the IC₅₀ for growth inhibition is lower on the resistant cells than on the corresponding parental cells, would be a hit. An example for this is vincristine and demecolcine in the 297/C5/0 and 298/D4/40 cells.

The cytotoxic agents and chemosensitizers of epothilones identified according to the methods of the present invention are particularly useful in a method to inhibit the growth of epothilone resistant cell lines. A method to inhibit growth of epothilone resistant cell lines using cytotoxic agents and chemosensitizers of epothilones would comprise contacting said epothilone resistant cells with one or more epothilones and one or more of the chemosensitizers. A method to inhibit growth of epothilone resistant cell lines using cytotoxic agents identified according to the methods of the present invention would comprise contacting said resistant cells with one or more cytotoxic agents. The term “contacting” is intended to include in vitro, ex vivo or in vivo administration. The cytotoxic agents and chemosensitizers identified according to the methods of the present invention may also be of therapeutic use in subjects bearing an epothilone resistant tumor (i.e., in situations where epothilones are not or no longer effective therapeutic agents due to natural or acquired resistance). Suitable pharmaceutical compositions of such cytotoxic agents and chemosensitizers, methods of administration and dosages would be apparent to one of skill in the art and could be performed according to conventional methodologies, including those discussed above.

The practice of the present invention will employ, unless otherwise indicated, conventional techniques of cell biology, molecular biology, cell culture, immunology and the like which are in the skill of one in the art. These techniques are fully disclosed in the current literature and reference is made specifically to Sambrook, Fritsch and Maniatis eds., “Molecular Cloning A Laboratory Manual, 2^{nd} edition, (Cold Spring Harbor Laboratory Press, 1989; the series Methods of Enzymology (Academic Press, Inc); and Antibodies: A Laboratory Manual, Harlow et al., eds., Cold Spring Harbor Laboratory Press (1987).

All publications, patents and patent applications mentioned in this specification are indicative of the level of skill of those skilled in the art to which this invention pertains.

The invention generally described above will be more readily understood by reference to the following examples, which are hereby included merely for the purpose of illustration of certain embodiments of the present invention and are not intended to limit the invention in any way.

### EXPERIMENTAL

The following materials and methods are used to conduct Examples 1-6:

Selection of Epothilone Resistant Cells

MDA-MB-435 cells are seeded (1×10⁶cells per plate) and allowed to grow in drug-free MEM supplied with 10% fetal calf serum, at 37° C. and 5.0% CO₂ to about 80% confluence. Growth of the cells is then continued in media containing 10 nm of epothilone A (Novartis AG, Basel, Switzerland) over 5 weeks with frequent media changes. Individual resistant colonies are isolated and successively expanded in 12 well, 6 well, 25 cm and 75 cm plates according to conventional methods. Resistant cells (founder cells) are then incubated in higher concentrations of epothilone to yield more highly resistant cell lines.

Growth Inhibition Assays

Growth inhibition may be analyzed using conventional methods, for example a MTS assay (PROMEGA, Wis.) may be used. In this assay approximately 4000 cells per well are seeded in 96 well plates in MEM supplemented with 10% fetal calf serum and incubated at 37° C. in 5.0% CO_{2.} After 24 hours, dilutions of drug (concentration range used is between 1 nM and 1000 nM) are added and cells are allowed to incubate with drug for an additional 72 hours. The MTS reagent mixed with PMS (MTS:PMS 20:1 according to manufacturer's instructions) is then added and absorbance quantitated using a Thermomax microplate reader system according to conventional methods (Molecular Devices, Sunnyvale, Calif.). Cells grown without drug for 24 hours are used as growth controls to determine the drug concentration resulting in 50% inhibition of cell growth (IC₅₀). Six assays per drug per plate are performed.

Western Immunoblotting

Plasma membrane proteins are extracted according to techniques known in the art (see, for example, Georges, E. et al. (1991) J. Cell Physiol. 148:479-484; Archiani-Matheis, A. et al. (1995) Oncol. Res. 12:603-610). Equal amounts of proteins are electrophoresed by SDS-PAGE, transferred onto PVDF membranes (Millipore, Mass.) and probed with monoclonal antibodies against P-gp and MRP according to Monks, A et al., JNCl, 83:757-766 (1991)).

RNA Isolation and Northern Blotting

Total RNA and poly A RNA are isolated from the various cell lines according to conventional methods (for example, compounds and methodology are commercially available from Qiagen, CA). Labelling of poly A RNA and hybridization of microarray chips are obtained commercially (Incyte Pharmaceuticals, Palo Alto, Calif.). Northern blotting is performed according to conventional methods and 10 μg of total RNA is electrophoresed over 1% agarose gels, transferred onto nylon membranes and hybridized with ³³P labelled probes synthesized from EST clones (Genome Systems, Inc., St. Louis Mo.). Northern blot signals are detected and quantified using phosphoimaging techniques according to conventional methods. cDNA probes containing the fluorescamine tagged nucleotides Cy3-dUTP or Cy5-dUTP are obtained from Incyte Pharmaceuticals, Palo Alto, Calif.

Cell Cycle Analysis

Cells are seeded on 10 cm²tissue culture dishes at a density of 1×10⁶ cells in MEM supplemented with 10% fetal calf serum and incubated overnight at 37° C. in 5.0% CO₂, exposed to drug (2× IC₅₀ concentration) for 24 hours, harvested by treatment with trypsin, collected by centrifugation and washed once with 10 ml of PBS (phosphobuffered saline). according to conventional methods. Cells are fixed by resuspending in cold 70% ethanol, washing again with 5 ml of PBS and resuspending in PI solution (70 μl propidium iodide, 38 mM sodium citrate, 20 μg/ml RNAse A). After incubation in PI solution at 37° C. for 30 minutes, cells are analyzed by flow cytometry according to conventional methodology (apparatus and software commercially available from Becton Dickinson Immunocytometry Systems, CA).

### EXAMPLE 1

### Generation of Epothilone A Resistant Cell Lines

Epothilone A resistant subclones of the parental MDA-MB-435 breast adenocarcinoma cell line are established according to methods described above. Specifically, MDA-MB-435 breast adenocarcinoma cells are incubated in MEM supplemented with 10% fetal calf serum in the presence of 10 nM of epothilone A at 37° C. under 5% CO₂ for 5 weeks. Although the majority of the cells die, surviving clones are selected and further expanded in media containing 10 nM epothilone A. One out of 12 resistant colonies expanded well and is designated herein as the EA10 cell line. EA10 cells are maintained in media containing 10 nM epothilone A. Continued culture of the EA10 cells (founder cells) in higher concentrations of epothilone A yield additional, more highly resistant cell lines; cell lines resistant to 20 nM (EA20), 40 nM (EA40), 60 nM (EA60) or 150 nM (EA150) epothilone A are isolated in this manner.

### EXAMPLE 2

### Epothilone A Resistant Cells Overcome Epothilone A-Induced G₂/M Block

Cell cycle distribution patterns of epothilone A resistant and parental MDA-MB-435 epothilone A sensitive cells are compared by standard FACS analysis. Data indicate that the process of cell division in most of the MDA-MB-435 cells cultured for 24 hours in the presence of 20 nM epothilone A is blocked at the G2/M phase of the cell cycle. In contrast, a large proportion of EA60 cells cultured for 24 hours in 60 nM epothilone A overcomes the G2/M phase block and reenter G1 and S phases. Thus, a significant part of the epothilone A resistant cell population appears to overcome the G2/M block that normally occurs in the cell cycle of epothilone A sensitive cells.

### EXAMPLE 3

### Two Potential Mechanisms of Resistance in Epothilone A Resistant Cells

The MTS assay is used to determine the concentration (IC₅₀ ) of drug required to inhibit the growth of 50% MDA-MB-435, EA10, EA20, EA40, EA60, and EA150 cells. As shown in Table 1, the IC₅₀ of epothilone A in EA10 and EA20 cells increases 5 fold and 7 fold respectively, over MDA-MB-435 cells. Interestingly, the IC₅₀ of epothilone A is essentially the same in EA20 and EA40 cells, although EA40 cells are selected in double the concentration of epothilone A as is used to select EA20 cells. However, a 15 fold and a >100 fold increase in the IC₅₀ of epothilone A in EA60 and EA150 cells, respectively, is observed. Furthermore, in contrast to the increasing resistance to epothilone A, EA40 and EA150 cells are equally resistant to TAXOL®. Thus, at least two potential mechanisms of epothilone A resistance may exist—a lower resistance mechanism that is cross-resistant to TAXOL® and a higher, epothilone A specific resistance mechanism.

TABLE 1
IC₅₀ Values of Epothilone A (EpoA), Epothilone B (Epo B) and TAXOL ® Compared
in MDA-MB-435, EA10, EA20, EA40, EA 60 and EA150 Cells

| | MDA-MB- | | | | | |
|---|---|---|---|---|---|---|
| | 435 | EA10 | EA20 | EA40 | EA60 | EA150 |
| Drug | IC₅₀/nM | IC₅₀/nM | IC₅₀/nM | IC₅₀/nM | IC₅₀/nM | IC₅₀/nM |
| EpoA | 4 ± 1.8 | 18 ± 3.8 | 28 ± 3.0 | 36 ± 2.0 | 82 ± 1.5 | >500 |
| EpoB | 2 ± 0.8 | 3 ± 1.4 | 6 ± 2.9 | 8 ± 3.5 | 10 ± 4.2 | 15 ± 3.1 |
| TAXOL ® | 7.5 ± 3.0 | 13 ± 3.1 | 22 ± 4.0 | 23 ± 4.0 | 27 ± 3.1 | 34 ± 2.6 |

### EXAMPLE 4

### P-gp or MRP Do not Mediate Resistance to Epothilone A

P-gp and/or MRP expression levels are analyzed in epothilone A sensitive and resistant cells by Western immunoblotting. Data indicate that, whereas multidrug resistant MDA/T0.3 cells and adryamicin resistant HL60 cells express P-gp and MRP, respectively, neither P-gp nor MRP are detected in sensitive MDA-MB-435 cells or the resistant EA10 or EA20 cells.

Since the epothilone A resistant cells disclosed herein exhibit limited cross resistance to TAXOL®, a P-gp substrate, the possibility exists that epothilone resistance is mediated by a level of P-gp too low to be detected by Western blotting. As such, epothilone resistant cells would have some degree of cross-resistance to other P-gp substrates that could be reversed by P-gp inhibitors. To investigate this possibility, the cytotoxicity of the P-gp substrates, vincristine, doxorubicine and vinblastine is determined against epothilone A resistant cells using the MTS assay. As shown in Table 2, the parental MDA-MB-435 cell lines and the epothilone A resistant EA40 cell line are equally sensitive to all three cytotoxic drugs. When EA20 and resistant MDA/T0.3 cells are incubated with TAXOL® or epothilone A in the presence of [3′-Desoxy-3′-oxo-MeBmt]¹-[Val]²-Ciclosporin (PSC-833; a P-gp inhibitor which is disclosed in U.S. Pat. No. 5,525,590), the resistance of MDA/T0.3 cells to TAXOL® is reversed but no effect on the resistance of EA20 to epothilone A is observed.

MDA/T0.3 is a clonal subline of MDA-MB-435 cells selected by increasing exposure of MDA-MB-435 cells to TAXOL®. MDA/T0.3 cells express high levels of P-glycoprotein and are resistant to a variety of chemically and functionally unrelated compounds. MDA/T0.3 cells could be sensitized to a variety of cytotoxic agents by treatment with the P-glycoprotein reversing agent PSC-833.

Taken together, these data indicate that P-gp or MRP does not mediate epothilone resistance nor are these drug efflux proteins upregulated in these cells.

TABLE 2
IC₅₀s of Various P-gp Substrates Compared
in MDA-MB-435, MDA/T0.3 and EA40 Cells

| | MDA-MB-435 Cells | MDA/T0.3 Cells | EA40 Cells |
|---|---|---|---|
| Drug | (IC₅₀/nM) | (IC₅₀/nM) | (IC₅₀/nM) |
| Taxol | 7.5 ± 3 | 540 ± 100 | 21 ± 3 |
| Vincristine | 3 ± 2 | — | 4 ± 1.7 |
| Vinblastin | <1 | 14 ± 2.7 | <1 |
| Daunorubicin | 382 ± 101 | >5000 | 224 ± 72 |
| Doxorubicin | >500 | >5000 | 365 ± 200 |

### EXAMPLE 5

### Differential Gene Expression Characteristic of Epothilone Resistant Cells

Conventional methods, including high-density cDNA microarrays, may be used to examine differential gene expression characteristic of the epothilone resistant cells. For example, RNA was isolated from MDA-MB-435, EA20, EA40 and EA150 cells and used to prepare cDNA probes that incorporate the fluorescamine tagged nucleotides cye3-dUTP or cye5-dUTP. Microarrays of 7500 nonredundant gene specific cDNA sequences on glass chips (commercially available from Incyte Pharmaceuticals, CA) are simultaneously hybridized with fluorescently tagged cDNA representations of total RNA pools from EA20, EA40 or EA150 cells as test samples and MDA-MB-435 as control (labelling and hybridization were done by Incyte Pharmaceuticals, Palo Alto, Calif.). For each hybridization, the test and control are tagged with a different flourescent label and for each cDNA spot, the relative amount of its representation in each pool is determined by the net flourescent signal generated at that spot according to methods known in the art (see, for example, Shena, M. et al. (1995) Science 270:467-470). Chips hybridized with cDNA from MDA-MB-435 and EA150 cells, each cDNA pool labelled with a different dye, result in several differentially expressed genes. Analysis of the gene expression data is performed using proprietary software, but conventional methodologies may also be used. Comparison of duplicate experiments indicate fluctuations in the levels of genes differentially expressed below 3 fold (data not shown); further analysis was performed on genes that are differentially expressed over 3 fold. To corroborate the microarray results, a subset of the differentially expressed genes are analyzed by Northern blotting according to conventional methods and differential expression of the subset of genes analyzed confirmed.

In order to ascertain that massive and nonspecific changes in gene expression did not occur during selection of epothilone A resistant cells, the proportion of genes changing by more than 3 fold is calculated. Of the 7500 genes arrayed on the chip, signals are detected from 4000 spots and the expression of only 1.8% changed. The same number of genes is differentially expressed in EA40 and EA150 cells.

To further distinguish genes that are differentially expressed specifically in epothilone resistant cells, microarray chips may be probed with cDNA synthesized from the MDA-MB-435 parental cell line as well as from the multidrug resistant MDA/T0.3 cell line that was selected in the presence of TAXOL®. Comparison of genes differentially expressed in MDA/T0.3 or in EA150 cells allows the identification of genes upregulated or repressed in both cell lines. Only a small percentage of the genes whose expression changed in EA150 cells also changed in MDA/T0.3 cells. Thus, most of the microarray hits are specific for epothilone A resistant cells.

A search of the NCBI database with the EST sequences present on the microarrays indicate that more than 50% of the genes upregulated in the epthilone A resistant cell lines encode known interferon-inducible proteins. A high proportion of these upregulated genes encode histocompatibility class II proteins (HLA II). Interestingly, the expression of a number of the upregulated genes change with increasing epothilone A resistance, including the HLA II proteins.

Most of the non-interferon inducible upregulated genes encode cell surface proteins or intracellular signalling proteins (Table 3). A few of the upregulated genes were also identified as microtubule associated or cytoskeletal proteins, drug metabolizing enzymes or stress proteins.

TABLE 3
Classification of Genes Upregulated in EA150 Cells According
to Biochemical Function or Site of Expression

| | Microtubule | Drug Metabolism | | |
|---|---|---|---|---|
| | Associated | or Drug | | |
| Cell Surface or | or other | Resistance | Signal | Stress or |
| Extracellular Matrix | Cytoskeletal | Associated | Transduction | Unclassified |
| HLA II SB-a | Microtubule | Metallothionein 1 | EGR1 | Human p27 |
| HLA II DR-a | Associated | | | Human p78 |
| HLA II DR-a | p44 | Expoxide | IFN induced | 5′ nucleotidase |
| Annexin | | hydrolase | leucine zipper | KIAA0018 |
| CD74 | Mx gene | | | NIL-2-A |
| IFN induced 56K | | LRP | STAT1 | 1-8U |
| HLA II DP-a | Vimentin | | P68 kinase | KIAA0438 |
| HLA II DR-b1 | | | Clk 1 | PD978433 |
| Transmenbrane 4 | | | HSPDE3B | EST |
| Superfamily Galectin | | | ELF4A | PD1901249 |
| HLA II DQ-b | | | Dead box | KIAA0438 |
| IGFBP10 | | | protein | Follistatin |
| HLA II DQ-b | | | TFIIB | DNA J homolog |
| HLA DP-b | | | HMGIC | Glutamin |
| Integrin-a 6 | | | Protein kinase | cyclotransferase |
| Tenascin C | | | inhibitor p58 | AHNAK |
| Integrin a 4 subunit | | | CAMP kinase | |
| Lectin 14 | | | R II-a | |
| | | | Phospholipase | |
| | | | Ca | |
| | | | CDC7 related | |
| | | | kinase | |

The pattern of gene expression characteristic of the epothilone A resistant cells disclosed herein, particularly that of the EA150 cell line, can be used to identify cells, including tumor cells, that may be epothilone resistant. For example, cDNA may be obtained from test cells and gene expression for these cells characterized according to conventional methods. A pattern of expression similar to that of the epothilone A resistant cells claimed herein (e.g. the EA150 cell line) would indicate that the test cell is potentially epothilone resistant.

The following materials and methods are employed to perform Examples 6-13:

Epothilones A and B are obtained by the Biomolecular Production Unit, Novartis AG via fermentation of myxobacteria Sorrangium cellulosum, followed by purification (e.g. Hofmann et al., WO9942602). Paclitaxel is from Calbiochem (La Jolla, Calif.). Discodermolide is obtained from S. Pomponi (Harbor Branch Oceanographic Institute, Fort Pierce, Fla.) but may also be obtained as in U.S. Pat. No. 5,010,099. Vinblastine sulfate (Velbe®) is purchased from Eli Lilly (Indianapolis, Ind.), while demecolcine and 5-Fluorouracil are obtained from SIGMA (St. Louis, Mo.). Paraplatin is from Bristol Myers Squibb (Princeton, N.J.).

Selection of Epothilone Resistant Cells

Human KB-31 (drug-sensitive) and KB-8511 (multidrug-resistant, P-gp (P-glycoprotein) over-expressing) epidermoid carcinoma cells are obtained from Dr. R. M. Baker, Roswell Park Memorial Institute (Buffalo, N.Y., USA) (also may be obtained through the ATCC) and cultured as previously described (Utz et al., Int. J. Cancer 57:104 (1984)). Briefly, cells are cultured with complete MEM-alpha medium (minimal essential medium-alpha complemented with 5% foetal bovine serum and 1% (v/v) L-glutamine) at 37° C. in a 5% (v/v) CO₂ and 80% relative humidity atmosphere.

KB-31 epidermoid carcinoma cells are seeded at 0.5×10⁶ cells per 10 cm dish and incubated for 3 days in complete MEM-alpha medium in the presence of 1.7 nM epothilone A. Following incubation for an additional 3 days in the presence of 5 nM epothilone A, cells are subcultured (split ratio 1:10) and expanded in the presence of 5 nM epothilone A. Surviving cells are then further selected by intermittent exposure to step-wise (2-fold) increased epothilone A concentrations. At several stages during this process, single cell colonies are removed from the 10 cm dishes by mechanical force and expanded in 24 well dishes, 25 cm and 75 cm flasks. Colonies remaining on the dish are pooled, reseeded in the presence of 2-fold higher concentrations of epothilone A and individual colonies expanded as described above. This selection procedure resulted in the subline termed EpoA320-80C, which is first selected in the intermittent presence of 320 nM epothilone A (included at every other media change/subculture) and which subsequently is adapted to the continuous growth in 80 nM epothilone A (included at every media change/subculture). This subline turned out to be partially dependent on the presence of epothilone A. However, we are able to isolate a subline of EpoA320-80C (probably harboring a compensating mutation) which grew in the absence of drug, which we termed EpoA320/ND. In order to assure the clonality of the sublines, they are submitted to an additional round of subcloning, resulting in the lines 298/D4/40 and 297/C5/0, derived from EpoA320-80C and EpoA320/ND, respectively. Optimal growth of 298/D4/40 occurs in the presence of 40 nM epothilone A, while 297/C5/0 grows in the absence of epothilone A. An analogous scheme is employed to select KB-31 cells growing in the presence of epothilone B (starting concentration 0.6 nM). The subclone with the highest degree of resistance, designated 315/sc5.9, was initially maintained in the presence of 2.0 nM epothilone B and then adapted to growth in the absence of epothilone B.

In vitro Cell Growth Inhibition Assay

Anti-proliferative assays are performed basically as described (Meyer et al., Int. J. Cancer 43:851 (1989)). Briefly, cells are seeded at 1.5×10³ cells/well into 96-well microtiter plates and allowed to adhere by incubation overnight. Compounds are added in serial dilutions on day 1. The plates are then incubated for an additional 3 days, after which the cells are fixed with 3.3% v/v glutaraldehyde, washed with water and stained with 0.05% w/v methylene blue according to conventional methods. After washing, the dye is eluted with 3% HCl and the optical density measured at 665 nm with a SpectraMax Plus (Molecular Devices, Sunnyvale, Calif.) according to manufacturers instructions. IC₅₀ values are determined by mathematical curve-fitting using the SoftPro2.6.1 program (Molecular Devices, Sunnyvale, Calif.) using the formula (OD treated−OD start)/(OD control−OD start)×100. The IC₅₀ is defined as the drug concentration that led to 50% inhibition of net cell growth at the end of the incubation period. IC₅₀ determinations with the cell line 298/D4/40 are performed in the presence of 40 nM epothilone A.

Cell-Based and Cell-Free Tubulin Polymerization Assay

Drug-induced changes in the ratios between unpolymerized and polymerized tubulin in intact and lysed cells, respectively, are assessed basically as described (Giannakakou et al, Int. J. Cancer 75: 57-63 (1998)). Briefly, for the assessment of cellular drug effects, 2×10⁵ cells are seeded in a volume of 1 ml to maximally 6 wells per 24-well plate. After incubation for 18-24 hrs at 37° C. to allow for cell attachment, test substances are added to the cell culture medium to achieve the desired concentrations, followed by incubation of the cells for 2 hrs at 37° C. Final vehicle concentration is 1%. One plate at a time is then transferred to a dimmed tissue-culture hood and cells are washed twice with 1 ml of room-temperature phosphate-buffered saline lacking Ca⁺⁺ and Mg⁺⁺ (PBS/O). After careful removal of remaining PBS/O, 100 μl of room-temperature Hypotonic Lysis Buffer A (HLB/A: 20 mM Tris-HCl (pH 6.8), 1 mM Mg₂Cl, 2 mM EGTA, 2 mM Pefabloc, 1 mM benzamidine, 10 μg/ml aprotinin, 10 μg/ml leupeptin, 0.5% Nonidet P-40) is added and plates are wrapped in aluminum foil to minimize artifacts due to light-driven tubulin polymerization. Following incubation for exactly 5 min at 37° C., cells are harvested by scraping and transferred to a room-temperature eppendorf tube. Harvested wells are washed once with 100 μl room temperature HLB/A and pooled with the corresponding initial samples. Tubes are vortexed for 5 seconds to aid cell lysis, followed by centrifugation for 10 min at room-temperature in a table-top centrifuge at 12′000×g to separate microtubules (pellet) from unpolymerized tubulin (supernatant). The supernatant is then carefully transferred to a fresh eppendorf tube and stored on ice until further processed. Meanwhile, the pelleted material is resuspended in 200 μl room-temperature Resuspension Buffer (RSB: 10 mM Tris-HCl (pH 7.5), 1.5 mM Mg₂Cl, 2 mM EGTA, 1 mM Pefabloc, 1 mM benzamidine, 10 μg/ml aprotinin, 10 μg/ml leupeptin, 0.5% Nonidet P-40). Solubilization of the pellet is aided by placing the eppendorf tube for 30 seconds in a sonicator waterbath. Supernatant and solubulized pellet samples are then complemented with 6× sample loading buffer (U. K. Laemmli (1999) Nature 227, 680-685) heated for 5 min at 95° C. and subjected to SDS-PAGE on 10% resolving/5% stacking polyacrilamide gels as described below.

For the assessment of drug effects on the ratio between unpolymerized and polymerized tubulin in a cell-free system, cells are lysed at the time of drug addition and processed as follows. Cells are seeded and incubated for 18-24 hrs as described above. One plate at a time is then transferred to a dimmed tissue-culture hood and cells are washed twice with 1 ml of room temperature PBS/O. After careful removal of remaining PBS/O, 200 μl of room-temperature HLB/A containing test substances at the desired concentration is added to the cells. Final vehicle concentration is 1%. The plates are wrapped in aluminum foil and incubated for 5 min at 37° C. Lysates are then transferred to eppendorf tubes, vortexed for 5 seconds, followed by centrifugation for 30 minutes at room-temperature in a table-top centrifuge at 12′000×g. Further processing of the samples is performed as described above.

Crude Subcellular Fractionation

Crude subcellular fractionation to separate cytosolic and membraneous (“particulate”) cellular constituents is performed essentially as described (Borner et al, J. Biol. Chem. 264:13902-909 (1989)). Briefly, 1×10⁶ cells are seeded into 10 cm dishes and incubated at 37° C. until 70-80% confluent. Following transfer to ice, medium is removed by aspiration and cells are washed twice with 10 ml ice-cold PBS/O. Following careful removal of remaining wash buffer, cells are harvested in 500 μl Hypotonic Lysis Buffer B (HLB/B: 25 mM Hepes (pH 7.5), 25 mM β-glycerophosphate, 2 mM EDTA (pH 7.4), 1 mM PMSF, 10 μg/ml aproptinin, 10 μg/ml leupeptin, 1 mM Na3VO4), transferred to an eppendorf tube, and placed on ice. Cells are sonified with 10 bursts using a Branson Sonifier 250 (50% duty cycle, power output setting 6), avoiding frothing of the solution. Sonified cell lysates are then centrifuged for 15 min at 12′00×g in a tabletop centrifuge pre-cooled to 4° C. Supernatants (=Cytosol) are then transferred to fresh eppendorf tubes and stored on ice until further processed. Meanwhile, the remaining pellet sample is carefully rinsed with 1 ml HLB/B. Following addition of 250 μl Triton Lysis Buffer (TLB: HLB/B supplemented with 1% Triton X-100), samples are sonified and centrifuged as described above. The resulting supernatants (=Membrane) are then transferred to fresh eppendorf tubes. Following determination of protein content (BCA Kit, PIERCE, Rockford, Ill.), equal protein amounts are resolved by SDS-PAGE (7% resolving/5% stacking polyacrylamide gel) and antigens detected by immunoblotting as described below.

Immunoblotting

Proteins are separated by SDS-PAGE, transferred onto PVDF membranes (Millipore) and probed with rabbit anti-P-gp antibodies (PC03, dilution 1:100) (Calbiochem, San Diego, Calif.); or mouse anti-beta-tubulin (tub2.1, dilution 1:1000) (SIGMA; St. Louis; Mo.). and the corresponding horseradish peroxidase-labelled secondary antibodies (dilution 1:10′000) (Amersham, Piscataway, N.J.). Immunecomplexes are detected using the enhanced chemiluminescence method according to manufacturer instructions (ECL) (Amersham, Piscataway, N.J.).

### EXAMPLE 6

### Generation of Epothilone Resistant Cell Lines

The epothilone resistant cell lines, EpoA320-80C, EpoA320/ND, 298/D4/40, 297/C5/0 and 315/sc5.9 are generated as disclosed above. Specifically, the proliferation of KB-31 cells is half-maximally inhibited in the presence of 3.2 nM epothilone A. The incubation of KB-31 epidermoid carcinoma cells on an intermittent schedule with step-wise increasing concentrations of epothilone A result in the emergence of cells that are able to grow in the presence of significantly increased concentrations of epothilone A. Thus, EpoA-320 cells grew in the presence of 320 nM epothilone A, when administered once a week with a 3 day drug-free intermittent period. A subclone, 298/D4/40, is adapted to grow in the presence of 40 nM epothilone A administered twice weekly (i.e. at every medium change). Interestingly, it was found that these cells not only survive in the presence of more than 10-times the concentration of epothilone A that would lead to a 50% decreased cell growth in the parental cells, but, moreover, are dependent on this amount of drug for optimal growth. Furthermore, when 298/D4/40 cells are deprived of epothilone A, they growth arrest in the G₂/M phase of the cell cycle and eventually die by apoptosis. Intriguingly, epothilone B and paclitaxel, two other microtubule-stabilizing agents, are able to substitute for epothilone A in supporting the growth of this cell line (a similar drug-dependent phenotype had already been noted for the EpoA-320-80C cells). This suggests that 298/D4/40 cells have acquired a mutation that compromises the functionality of its tubulin in a fashion that can be complemented by other microtubule-stabilizing agents. Thus, this cell line could be employed in a drug screen, in which compounds with a potential microtubule-stabilizing mechanism could be identified by virtue of supporting the growth of epothilone A-deprived 298/D4/40 cells (see below). Nevertheless, upon prolonged incubation in the absence of epothilone A, we are able to isolate a subline that is still partially resistant to epothilone A, but has lost the drug-dependent phenotype of 298/D4/40, likely due to a cornpensating mutation. A subclone of this cell line, 297/C5/0, is used for further analyses.

The fact that the epothilone A can be replaced with epothilone B or paclitaxel (TAXOL®) to support the growth of the epothilone A-resistant/dependent cell line 298/D4/40 indicates that this cell line can be employed in a drug screen to identify compounds with a mechanism of action similar to that of epothilone A. To do so, one could incubate 298/D4/40 cells (which are previously deprived of epothilone A) with increasing concentrations of test compounds, followed by determination of cell numbers at the end of a 3 day. incubation period according to conventional methods. Potential hits would be those that support the growth of 298/D4/40 cells similar to epothilone A (or paclitaxel) that could be included as a positive control in this assay.

### EXAMPLE 7

### Drug Resistance Profile of 298/D4/40 and 297/C5/0 Cells

In a first set of analyses, the drug-resistance profile of 298/D4/40 and 297/C5/0 is determined (see Table 1). Both epothilone A-selected cell lines are either partially or fully cross-resistant to epothilone B and paclitaxel but intriguingly not cross-resistant to another microtubule-stabilizing agent, discodermolide. On the other hand, 298/D4/40 and, to a lesser extent, 297/C5/0, appear to be hypersensitive to the microtubule-destabilizing agents vinblastine and demecolcine. Neither cell line is cross-resistant to the standard cytotoxic drugs doxorubicine, 5-Fluorouracil or paraplatin (except for a very minor increase in the resistance to paraplatin noted for 298/D4/40). In contrast, the P-glycoprotein overexpressing KB-8511 cells are resistant to paclitaxel, vinblastine, demecolcine and doxorubicine, and thus display a classical multi-drug resistance profile. Taken together, the resistance profile observed for 292/D4/40 and 297/C5/0 is distinct from that of classical multi-drug-resistance.

TABLE 4
Cross-resistance profile of epothilone-selected KB-31 sublines

| | | Cell line (Selecting agent) | | | | | | | |
|---|---|---|---|---|---|---|---|---|---|
| | | KB-8511 | | 315/sc5.9 | | 297/C5/0 | | 298/D4/40 | |
| | | (Colch) | | (EpoB) | | (EpoA) | | (EpoA) | |
| Compound Class | Name | RF | SD | RF | SD | RF | SD | RF | SD |
| Microtubule stabilizer | Epothilone A | 0.7 | 0.3 | 1.2 | 0.0 | 44.7 | 24.2 | 71.4 | 9.2 |
| | Epothilone B | 0.9 | 0.3 | 3.1 | 0.3 | 7.7 | 2.3 | 9.2 | 1.1 |
| | Paclitaxel | 209.6 | 40.8 | 1.4 | 0.1 | 98.3 | 35.7 | 200.1 | 23.6 |
| | Discodermolide | 2.8 | 0.9 | 1.2 | 0.1 | 1.3 | 0.3 | 0.5 | 0.0 |
| Microtubule destabilizer | Vinblastine | 162.4 | na | 0.6 | 0.0 | 0.7 | 0.1 | 0.2 | 0.0 |
| | Demecolcine | 6.2 | 0.1 | 0.7 | 0.0 | 0.7 | 0.1 | 0.5 | 0.1 |
| Topoisomerase Inhibitor | Doxorubicine | 48.8 | 15.1 | 1.8 | 0.3 | 1.6 | 0.4 | 1.2 | 0.1 |
| Anti-metabolite | 5-FU | 1.1 | 0.2 | 0.4 | 0.1 | 1.2 | 0.8 | 0.9 | 0.2 |
| Alkylating agent | Paraplatine | 0.8 | 0.2 | 0.7 | 0.2 | 1.2 | 0.5 | 3.8 | 1.2 |

Data in Table 4 are gathered according to the following methods: Cells (1×10³) are seeded in 96-well plates and allowed to adhere for 24 hrs. Compounds are then added as two-fold serial dilutions and incubation continued for an additional 72 hrs. Cell numbers at the beginning and at the end of the experiment are estimated by methylene blue staining of cellular protein mass according to conventional methods. The net mass increase of vehicle-treated cells is set as 100% growth. The IC₅₀ is determined as the drug concentration that leads to half-maximal growth inhibition compared to the control. The resistance factor (RF) for a particular drug is calculated by dividing the IC₅₀ determined on the resistant cell line by that determined on the parental KB-31 cells. Data are presented as average±standard deviation (SD) of three separate experiments, except na=not applicable (n<3). Colchicine-selected KB-8511 cells overexpressing P-glycoprotein served as a reference for the classical multi-drug-resistant phenotype.

The KB-31 subline 315/sc5.9, which is selected in the presence of epothilone B, displayed 3.1-fold resistance towards epothilone B, and marginal cross-resistance to epothilone A, paclitaxel and doxorubicine. Similar to 298/D4/40, 315/sc5.9 is slightly hypersensitive towards the microtubule-destabilizing agents vincristine and demecolcine, as well as against a non-microtubule targeting drug, 5-fluorouracil. The significantly lower general resistance levels achieved for this clone compared to that of the epothilone A-selected clones 292/D4/40 and 297/C5/0 is unclear, but might be a consequence of the selection schedule chosen (step-wise 2-fold concentration increases).

### EXAMPLE 8

### P-gp Does not Mediate Epothilone Resistance in 315/sc5.9, 298/D4/40 and 297/C5/0 Cells

In order to confirm that epothilone-resistance does not involve P-glycoprotein, cell lysates enriched for membrane proteins are assessed for the presence of P-glycoprotein by immunoblotting. Specifically, cells (2×10⁶) are seeded in 15 cm dishes and grown until 70-80% confluent. Cells are harvested in hypotonic lysis buffer and subjected to crude subcellular fractionation by centrifugation as described above. The pellet constituting the particulate fraction (enriched in plasma membranes) is resuspended in a conventional detergent buffer. Equal protein amounts (10 μg) are resolved by SDS-PAGE, transferred to PVDF membranes, and P-glycoprotein detected using a commercial antibody (mdr-1 (P-glycoproetin) (Ab-1), Calbiochem-Novachem, San Diego, Calif.) using the ECL system. Particulate fraction of colchicine-selected KB-8511 cells (overexpressing P-glycoprotein) serves as a positive control.

Results indicate that, like the parental KB-31 cells, no P-gp expression is detectable in 315/sc5.9, 298/D4/40 and 297/C5/0 cells. In contrast, the “classical” multi-drug-resistant KB-8511 cells, which are derived from KB-31 cells by selection with colchicine (Akiyam et al, Som. Cell Mol Gen. 11:117-126 (1985)), overexpresses P-gylcoprotein. Taken together with the fact that epothilones A and B retain full activity against KB-8511 cells (see Table 4), these observation not only strongly suggests that epothilone-resistance does not involve P-glycoprotein, but also that repeated exposure of cells with these drugs does not induce this drug efflux pump.

### EXAMPLE 9

### Tubulin Polymerization in 298/D4/40 or 297/C5/0 Cells

Besides mechanisms that lead to a decreased cellular accumulation or increased metabolic inactivation of a particular drug, alterations in the target gene are also frequently observed. Thus, we tested whether 298/D4/40 or 297/C5/0 cells have a decreased tubulin polymerization potential when exposed to epothilone A. Specifically, KB-31, 297/C5/0 and 298/D4/40 cells (2×10⁵) are separately seeded in 6-well plates and allowed to adhere for 24 hrs. Cell are incubated for 2 hours with 0, 1, 10, 100 or 1000 nM concentrations of epothilone A or vehicle (0.1% DMSO). Treated cells are then harvested in hypotonic lysis buffer and soluble tubulin (present in the supernatant) separated from microtubule polymers (present in the pellet) by centrifugation as described above. The amount of unpolymerized tubulin is estimated by resolving a fraction of the supematant protein by SDS-PAGE followed by immunodetection of the blotted proteins using the β-tubulin pecific antibody TUB 2.1 (Sigma, St. Louis, Mo.) and the ECL system according to manufacturers instructions.

Results indicate that incubation of the parental KB-31 cells with increasing concentrations of epothilone A lead to a dose-dependent decrease in the unpolymerized pool of tubulin in these cells, with a concomitant increase in the pool of polymerized microtubules (data not shown). Interestingly, the concentrations of epothilone A which lead to a decrease in unpolymerized tubulin is in a similar range as the IC₅₀ for growth inhibition (see Table 4). In contrast, the drug concentrations required to decrease the unpolymerized tubulin fraction in 297/C5/0 cells are significantly higher, whereas 298/D4/40 cells are completely refractory to epothilone A-mediated tubulin polymerization in the dose-range tested (i.e. up to 1000 nM). However, it was not clear from this experiment whether the decrease in epothilone A-mediated tubulin polymerization is due to decreased drug uptake or a genuine defect in tubulin functionality. To address this question, cells are lysed before incubation with epothilone A. Specifically, KB-31, 297/C5/0 and 298/D4/40 cells (2×10⁵) are separately seeded in 6-well plates and allowed to adhere for 24 hrs. Cells are then lysed in hypotonic buffer containing 0, 1, 10 or 100 μM epothilone A. Vehicle (DMSO) concentration was 1%. Following incubation for 5 min at 37° C., samples are centrifuged to separate soluble from polymerized tubulin and the levels of unpolymerized tubulin is assessed by Western blotting as described above.

Results indicate that increasing concentrations of epothilone A lead to a dose-dependent decrease in the unpolymerized pool of tubulin in KB-31 cell lysates. The dose-response curve for 297/C5/0 cells is shifted to higher concentrations, while the tubulin pool in 298/D4/40 is again completely refractory to epothilone A-mediated changes in the levels of unpolymerized tubulin. It is also noteworthy that the concentrations of epothilone A required to elicit an effect in this cell-free assay format are several orders of magnitude higher than that observed for the cell-based assay. This is in agreement with eariler observations that epothilones (as well as paclitaxel) accumulate several-hundred fold inside cells. Taken together, these results suggest that 297/C5/0 and 298/D4/40 cells have acquired a mutation, or several mutations, that directly or indirectly alter their tubulin functionality.

Nucleotide sequence determination of the HM40 β-tubulin isotype revealed the presence of a single base mutation present in 297/C5/0 and 298/D4/40 cells when compared with the parental KB-31. Thus, nucleotide 820, the first nucleotide in codon 274, was changed from A→C. The altered codon (ACC→CCC) results in a non-conservative amino acid change from threonine to proline (Thr274Pro). Standard molecular biology methods are employed. Briefly, overlapping fragments covering the entire coding sequence are amplified from total RNA by RT-PCR and sequenced by automated cycle PCR using the ABI PRISM® BigDye™ Terminator Cycle sequencing kit (PE Biosystems). The four isoform-specific primer sets used have been reported previously (Giannakakou et al., J. Biol. Chem. 272(27): 17118-17125 (1997)).

1. An epothilone resistant cell line derived from the MDA-MB-435 breast adenocarcinoma cell line.

2. The cell line of claim 1, wherein said cell line is resistant to 10 nM epothilone A.

3. The cell line of claim 1, wherein said cell line is resistant to 20 nM epothilone A and is the EA20 cell line deposited under the Patent Deposit Designation PTA-2702.

4. The cell line of claim 1, wherein said cell line is resistant to 40 nM epothilone A and is the EA40 cell line deposited under the Patent Deposit Designation PTA-2708.

5. The cell line of claim 1, wherein said cell line is resistant to 60 nM epothilone A and is the EA60 cell line deposited under the Patent Deposit Designation PTA-2703.

6. The cell line of claim 1, wherein said cell line is resistant to 150 nM epothilone A and is the EA150 cell line deposited under the Patent Deposit Designation PTA-2706.

7. An epothilone resistant cell line derived from the KB-31 carcinoma cell line.

8. The cell line of claim 7, wherein said cell line is the carcinoma cell line KB-31/298 deposited under the Patent Deposit Designation PTA-2705.

9. The cell line of claim 7, wherein said cell line is the carcinoma cell line KB-31/297 deposited under the Patent Deposit Designation PTA-2704.

10. The cell line of claim 7, wherein said cell line is the carcinoma cell line KB-31/315 deposited under the Patent Deposit Designation PTA-2707.

11. A method to identify agents which display improved cytotoxicity to epothilone resistant cells in comparison to epothilone, said method comprising the steps of:
a) incubating said epothilone resistant cells and sensitive cells with an agent to be tested;
b) determining the cytotoxicity of said agent for epothilone resistant cells and sensitive cells; and,
c) identifying agents which display a reduced resistance factor (IC₅₀ value for resistant cells divided by IC₅₀ value for parental cells) compared to epothilone.

12. A method to identify agents which display selective cytotoxicity to epothilone resistant cells in comparison to epothilone, said method comprising the steps of:
a) incubating said epothilone resistant cells and sensitive cells with an agent to be tested;
b) determining the cytotoxicity of said agent for epothilone resistant cells and sensitive cells; and,
c) identifying agents which display a resistance factor (IC₅₀ value for resistant cells divided by IC₅₀ value for parental cells)<1.

13. A method to inhibit the growth of epothilone resistant cells in vitro or in vivo, said method comprising contacting said resistant cells with one or more of said cytotoxic agents of claim 12.

14. A method to identify agents which are chemosensitizers of epothilones, said method comprising the steps of:
a) incubating epothilone resistant cells with an epothilone to which the cells are resistant in the presence and absence of an agent to be tested; and,
b) determining the cytotoxicity of the epothilone for the cells, wherein increased cytotoxicity in cultures incubated with the agent compared to cultures incubated in the absence of the agent indicates that the agent is a chemosensitizer.

15. The chemosensitizers of epothilones identified according to the method of claim 14.

16. A method to inhibit the growth of epothilone resistant cells in vitro or in vivo, said method comprising contacting said epothilone resistant cells with one or more epothilones and one or more of the chemosensitizers identified according to the method of claim 14.

17. A method to identify potential epothilone resistant cells, including tumor cells, said method comprising the steps of:
a) preparing total mRNA from test cells and corresponding normal cells or tumor and non-diseased tissue, as well as from control epothilone resistant cells and parental epothilone-sensitive cells;
b) preparing cDNA from the total mRNA of said cells or tissues;
c) performing differential gene expression analysis of said cDNA samples; and,
d) comparing the differential expression patterns observed for said test cells or tumors vs. their corresponding controls, with that of said epothilone resistant cells vs. the parental epothilone-sensitive cells, wherein similar differential gene expression patterns suggest that the test cells are potentially epothilone resistant cells.

18. Purified antibodies specific for the epothilone resistant cells in claim 1.

19. A pharmaceutical composition comprising one or more purified antibodies according to claim 18 in conjunction with a pharmaceutically acceptable carrier or diluent.

20. A method of identifying epothilone resistant cell lines, said method comprising the steps of:
a) labelling one or more antibodies according to claim 18;
b) exposing test cells to said one or more labelled antibodies; and,
c) measuring binding of said one or more labelled antibodies thereto.

21. A method of isolating epothilone resistant cell lines, said method comprising:
a) labelling one or more antibodies of claim 18 with a fluorescent dye;
b) incubating cell mixtures with said one or more fluorescently labelled antibodies; and,
c) isolating epothilone resistant cells fluorescently labelled with one or more antibodies by use of a fluorescence activated cell sorter.

22. A method for killing epothilone resistant cells, said method comprising:
a) attaching a substance having toxic or therapeutic activity to one or more antibodies of claim 18;
b) administering said one or more antibodies to cell culture (or a patient); and,
c) targeting the cytotoxic drug to epothilone resistant cells, selectively killing these cells.

23. A method for diagnosing an epothilone resistant cancer in vivo, comprising:
a) labelling one or more of the antibodies of claim 18;
b) administering said one or more labelled antibodies to a patient; and,
c) identifying tissue to which said one or more labelled antibodies bind.

24. A method of treating cancer, said method comprising administering to a subject in need thereof a therapeutically effective amount of one or more of the antibodies identified according to claim 18.

25. A method to identify epothilone analogs, said method comprising the steps of:
a) contacting an epothilone resistant cell line of claim 1 and the corresponding parental cell line with increasing concentrations of a test substance; and,
b) determining resistance of the epothilone resistant cell line to the test substance compared to resistance of the parental cell line to the test substance, wherein greater resistance to the test substance of the epothilone resistant cell line compared to the parental cell line suggests that the test substance is an epothilone analog.

26. The epothilone analogs identified according to the method of claim 25.

27. A method to identify compounds which are potential microtubule stabilizing agents said method comprising the steps of:
a) incubating epothilone-dependent cells with a compound to be tested wherein said resistant cells are epothilone deprived; and,
b) assaying for growth of said dependent cells, wherein growth of said dependent cells would indicate that said compound is a potential microtubule-stabilizing agent.

28. The microtubule stabilizing agents identified according to the method of claim 27.
